(19) United States
(12) Patent Application Publication
(10) Pub. No.: US 2015/0071868 A1
(43) Pub. Date: Mar. 12, 2015

(54) PRODUCT FOR TEMPORARILY DEFORMING KERATINOUS FIBERS ON THE BASIS OF A COMBINATION OF SPECIFIC FILM-FORMING POLYMERS

(71) Applicant: Henkel AG & Co. KGaA, Dusseldorf (DE)

(72) Inventors: Diane Metten, Hamburg (DE); Bernd Richters, Hamburg (DE)

(21) Appl. No.: 14/367,057

(22) PCT Filed: Nov. 5, 2012
(86) PCT No.: PCT/EP2012/071798

§ 371 Date: Jun. 19, 2014

(30) Foreign Application Priority Data
Dec. 20, 2011 (DE) 10 2011 089 171.4

(51) Int. Cl. A61K 8/81; A61Q 5/06

(52) U.S. Cl. CPC A61K 8/8182; A61K 8/8152; A61K 8/8158; A61Q 5/06; A61K 2800/54
USPC 424/70.16

(57) ABSTRACT

Cosmetic agents particularly suitable for temporarily deforming keratinic fibers are provided that contain in a cosmetically acceptable carrier

a) a copolymer A formed from
a monomer (A1) from the group of unsaturated carboxylic acids and unsaturated carboxylic acid esters,
a monomer (A2) from the group of unsaturated amine group-containing monomers,
a monomer (A3) from the group of unsaturated amphiphilic monomers,
   b) a copolymer B formed from
a monomer (B1) selected from quaternized N-vinylimidazoles,
a monomer (B2) selected from N-vinyllactams are.

### CROSS REFERENCE TO RELATED APPLICATION

The present disclosure is a U.S. National Stage entry under 35 U.S.C. §371 based on International Application No. PCT/EP2012/071798, filed Nov. 5, 2012 which was published under PCT Article 21(2) and which claims priority to German Patent Application No. DE 10 2011 089 171.4 filed on Dec. 20, 2011, which is incorporated herein by reference in its entirety.

### TECHNICAL FIELD

The technical field describes cosmetic agents based on a specific polymer combination, use of these cosmetic agents for temporarily deforming keratinic fibers and cosmetic methods using these agents.

### BACKGROUND

Polymers are widely used in the most varied cosmetic agents. They are to be found in agents for treating skin as well as in agents for treating hair, in agents which are washed off or out again directly after use, i.e. “rinse-off products”, and in agents which remain on the skin or hair, i.e. “leave-on agents”. The polymers are used for the most varied reasons and specific properties of the polymers are exploited in each case. In agents for treating skin, in shampoos, hair rinses and hair masks, the emphasis often lies on the thickening or conditioning properties of the polymers. In agents for temporarily deforming keratinic fibers, hereinafter also known as styling agents, alongside these properties film-forming and/or setting effects are particularly desired. Polymers often also serve as auxiliaries for improving or indeed enabling deposition and fixing of other active substances and ingredients on the skin or hair. By adding suitable polymers to hair coloring agents, for example, rubbing fastness and coloring durability may be increased.

Cosmetic agents generally contain individual polymers which are specifically tailored to achieving a very specific effect. If various effects are to be achieved, a plurality of polymers must be added. However, using too many different polymers may be associated with a series of disadvantages. Problems may accordingly arise during formulation, for instance because the polymers react with one another or with other components of the agent resulting in precipitation or decomposition phenomena. Certain polymers also have a tendency to be deposited so permanently on the skin and in particular on the hair that they are no longer completely removed with normal washing and the polymer accumulates undesirably so ultimately leading to contamination of the skin or hair.

There is therefore a constant need for polymers or suitable combinations of small numbers of polymers which simultaneously exhibit as many as possible of the desired properties.

For example, in the case of styling agents, the polymers used need to give the treated hair the strongest possible hold. In addition to a high degree of hold, styling agents must meet a whole series of further requirements. These may be broadly divided into properties on the hair, properties of the respective formulation, for example properties of the foam, the gel or the sprayed aerosol, and properties which affect the handling of the styling agent, wherein properties on the hair are of particular importance. Particular mention should be made of moisture resistance, low tackiness and a well-balanced conditioning effect. Moreover, a styling agent should as far as possible be universally applicable for all hair types. If the styling agent is a gel or a paste, the polymers should additionally have thickening properties.

Accordingly, at least one object herein is to provide suitable polymer combinations that are distinguished by good film-forming and/or setting properties, have a very high level of hold without having to sacrifice flexibility and good moisture resistance, in particular perspiration and water resistance, and are additionally suitable for producing stably viscous and stably transparent cosmetic compositions. Furthermore, other desirable features and characteristics will become apparent from the subsequent detailed description of the invention and the appended claims, taken in conjunction with this background of the invention.

### SUMMARY

In accordance with exemplary embodiments, a specific polymer combination is provided. In a first exemplary embodiment a cosmetic agent contains in a cosmetically acceptable carrier

a) a copolymer A formed from
a monomer (A1) from the group of unsaturated carboxylic acids and unsaturated carboxylic acid esters,
a monomer (A2) from the group of unsaturated amine group-containing monomers,
a monomer (A3) from the group of unsaturated amphiphilic monomers
   b) a copolymer B formed from
a monomer (B1) selected from quaternized N-vinylimidazoles,
a monomer (B2) selected from N-vinyllactams.

The agents contemplated herein contain the active substances in a cosmetic carrier. For the purposes herein, said cosmetic carrier is aqueous, alcoholic or aqueous-alcoholic. The aqueous-alcoholic carriers should be taken to be hydrous compositions containing about 3 to about 70 wt. % of a C₁-C₄ alcohol, in particular ethanol or isopropanol, relative to the total weight of the mixture for use. For the purposes herein, an aqueous carrier contains at least about 30 wt. %, in particular at least about 50 wt. % water, relative to the total weight of the mixture for use. Preferred cosmetic agents contain, relative to the total weight thereof, about 40 to about 99 wt. %, preferably about 50 to about 98 wt. %, more preferably about 60 to about 95 wt. % and in particular about 70 to about 90 wt. % water. The pH value (about 10% solution, about 20° C.) of preferred cosmetic agent amounts to about 2 to about 9, preferably about 3 to about 7 and in particular about 4 to about 6.

The cosmetic agents contemplated herein contains as their first significant essential component a copolymer A formed from
a monomer (A1) from the group of unsaturated carboxylic acids and unsaturated carboxylic acid esters,
a monomer (A2) from the group of unsaturated amine group-containing monomers,
a monomer (A3) from the group of unsaturated amphiphilic monomers.

Preferred copolymers A are based on a monomer (A1) from the group acrylic acid, methacrylic acid, C₁-C₆ alkyl acrylic acid esters, C₁-C₆ alkyl methacrylic acid esters. The acrylate copolymers and methacrylate copolymers are preferably esters of the particular acids with non-tertiary alkyl alcohols having alkyl residues with 1 to 12 carbon atoms, in particular 2 to 4 carbon atoms. Suitable monomers which may be mentioned by way of example are ethyl acrylate, ethyl methacrylate, propyl acrylate, propyl methacrylate, n-butyl acrylate, n-butyl methacrylate, isobutyl acrylate, 2-methylbutyl acrylate, 2-ethylhexyl acrylate, n-octyl acrylate, isooctyl acrylate, isooctyl methacrylate, isononyl acrylate and isodecyl acrylate.

Monomers preferably used as monomer (A2) are from the group acrylamide, methacrylamide, mono-(C₁-C₄)-alkylamino-(C₁-C₄)-alkyl acrylate, di-(C₁-C₄)-alkylamino-(C₁-C₄)-alkyl acrylate, mono-(C₁-C₄)-alkylamino-(C₁-C₄)-alkyl methacrylate, and di-(C₁-C₄)-alkylamino-(C₁-C₄)-alkyl methacrylate. Exemplary, preferred monomers (A2) are 2-(N,N-dimethylamino)ethyl acrylate, 2-(N,N-dimethylamino)ethyl methacrylate, 2-(N,N-diethylamino)ethyl acrylate, 2-(N,N-diethylamino)ethyl methacrylate, 3-(N,N-dimethylamino)propyl acrylate, 3-(N,N-dimethylamino)propyl methacrylate, 2-(N,N-dimethylamino)neopentyl acrylate, N′-(3-N,N-dimethylamino)propyl acrylamide, and N′-(3-N,N-dimethylamino)propyl methacrylamide.

The group of amphiphilic monomers (A3) denotes monomers which have hydrophilic and hydrophobic substructures. Preferred monomers (A3) may in turn be based on three structural units:
an unsaturated acid, preferably acrylic acid, methacrylic acid or itaconic acid;
a polyoxyalkylene group, preferably a polyethylene glycol group, a polypropylene glycol group or a polyethylene glycol/polypropylene glycol group;
a C₈₋₄₀ alkyl chain, preferably a C₁₀₋₃₀ alkyl chain.

C₁₀-C₃₀ alkyl PEG 20-25 methacrylates, for example, are used as monomer (A3). However, the use of C₁₀₋₃₀ alkyl PEG itaconates, in particular C₁₀₋₃₀ alkyl PEG-20 itaconate is more preferably used as monomer (A3).

In summary, preferred copolymers A are those which are formed from
at least one monomer (A1) from the group acrylic acid, C₁-C₆ alkyl acrylic acid esters, C₁-C₆ alkyl methacrylic acid esters,
at least one monomer (A2) from the group acrylamide, methacrylamide, mono-(C₁-C₄)-alkylamino-(C₁-C₄)-alkyl acrylate, di-(C₁-C₄)-alkylamino-(C₁-C₄)-alkyl acrylate, mono-(C₁-C₄)-alkylamino-(C₁-C₄)-alkyl methacrylate, di-(C₁-C₄)-alkylamino-(C₁-C₄)-alkyl methacrylate,
at least one monomer (A3) from the group of C₁₀₋₃₀ alkyl PEG-20 itaconates.

Particularly preferred copolymers A are those which are formed from
a monomer (A1) from the group acrylic acid, methacrylic acid, C₁-C₆ alkyl acrylic acid esters, C₁-C₆ alkyl methacrylic acid esters,
a monomer (A2) from the group acrylamide, methacrylamide, mono-(C₁-C₄)-alkylamino-(C₁-C₄)-alkyl acrylate, di-(C₁-C₄)-alkylamino-(C₁-C₄)-alkyl acrylate, mono-(C₁-C₄)-alkylamino-(C₁-C₄)-alkyl methacrylate, di-(C₁-C₄)-alkylamino-(C₁-C₄)-alkyl methacrylate,
a monomer (A3) from the group of C₁₀₋₃₀ alkyl PEG-20 itaconates.

With regard to the cosmetic properties of the agents contemplated herein, it has proven advantageous for copolymer A to consist in a proportion of at least about 70 wt. %, preferably at least about 80 wt. %, preferably at least about 90 wt. % and in particular at least about 95 wt. % of the structural units (A1), (A2) and (A3).

Corresponding copolymers are designated according to INCI nomenclature as Acrylates/Aminoacrylates/C10-C30 Alkyl PEG-20 Itaconates and can be obtained for example from National Starch under the trade name Structure® Plus.

The proportion by weight of copolymer A in the total weight of cosmetic agents contemplated herein preferably amounts to about 0.05 to about 10 wt. %, preferably about 0.1 to about 7.0 wt. % and in particular about 0.2 to about 5.0 wt. %.

The agents contemplated herein contain as second essential component at least one copolymer B formed from
at least one monomer (B1) selected from quaternized N-vinylimidazoles,
at least one monomer (B2) selected from N-vinyllactams.

More preferred agents are characterized in that they contain a copolymer B which
contains a structural unit according to formula (B-I), in which
   R means a C₁ to C₃₀ alkyl group, a C₁ to C₄ aralkyl group, a C₂ to C₆ alkenyl group or a C₂ to C₆ hydroxyalkyl group and
   X⁻ denotes a physiologically acceptable anion
and contains a further structural unit according to formula (B-II), in which n denotes 1, 2 or 3 as the number of methyl units.

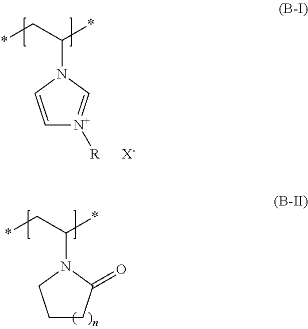

Film-forming and/or setting copolymers B are known. These copolymers have a structural unit according to formula (B-I) and a structural unit according to formula (B-II) and may additionally comprise further structural units, which are incorporated by polymerization through the addition of corresponding monomers during polymerization.

In formula (B-I), R denotes a C₁ to C₃₀ alkyl group, a C₁ to C₄ aralkyl group, a C₂ to C₆ alkenyl group or a C₂ to C₆ hydroxyalkyl group. Examples of preferred groups R are —CH₃; —CH₂CH₃, —CH₂CH₂CH₃, CH(CH₃)₂, —(CH₂)₃CH₃,

—CH₂—CH(CH₃)₂, CH(CH₃)CH₂CH₃, —C(CH₃)₃, —CH₂OH, —CH₂CH₂OH,

### —CH₂CH₂CH₂OH, —CH(OH)CH₂CH₃, —CH₂CH(OH)CH₃.

X⁻ denotes a physiologically acceptable anion, preferred anions being chloride, bromide, iodide, sulfate, methosulfate, ethyl sulfate, tosylate and tetrafluoroborate. More preferred agents are characterized in that the monomer (B1) is a salt of 3-alkyl-1-vinylimidazolium with physiologically acceptable anions, preferably 3-methyl-1-vinylimidazolium methylsulfate.

In formula (B-II), n denotes the number of methylene groups. With n=1 formula (B-II) denotes a vinylpyrrolidone unit, with n=2 it denotes a vinylpiperidinone unit and with n=3 it denotes a vinylcaprolactam unit. More preferred agents are characterized in that monomer (B2) is selected from N-vinylcaprolactam and N-vinylpyrrolidone.

More preferred agents contemplated herein are characterized in that they contain as copolymer B a copolymer B1 which
contains a structural unit according to formula (B-I), in which R denotes a methyl group and X denotes methosulfate,
contains a further structural units according to formula (B-II), in which n denotes 1 methylene unit.

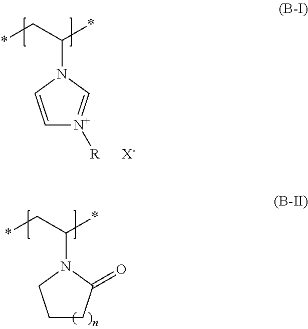

Particularly preferred copolymers B1 contain about 10 to about 30 mol %, preferably about 15 to about 25 mol % and in particular about 20 mol % structural units according to formula (B-I) and about 70 to about 90 mol %, preferably about 75 to about 85 mol % and in particular about 80 mol % structural units according to formula (B-II).

In this case, it is more preferred for the copolymers B1 to contain, in addition to polymer units which arise from the incorporation of the stated structural units according to formula (B-I) and (B-II) into the copolymer, at most about 5 wt. %, preferably at most about 1 wt. %, of polymer units originating from the incorporation of other monomers. The copolymers B1 are preferably exclusively synthesized from structural units of formulae (B-I) and (B-II) and may be described by the general formula

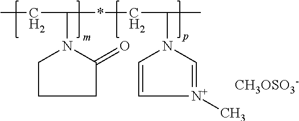

wherein the indices m and n vary depending on the molar mass of the polymer and are not intended to mean that the copolymers are block copolymers. Instead, structural units of the formula (B-I) and the formula (B-II) may be present randomly distributed in the molecule.

Such N-methylvinylimidazole/vinylpyrrolidone copolymers are designated POLYQUATERNIUM-44 according to NCI nomenclature and are obtainable for example from BASF under the trade name Luviquat® UltraCare.

More preferred agents contain a copolymer B1 which has molar masses within a specific range. Hair deforming agents are here preferred in which the copolymer B1 has a molar mass of about 50 to about 400 kDa, preferably of about 100 to about 300 kDa, more preferably of about 150 to about 250 kDa and in particular of about 190 to about 210 kDa.

In addition to the copolymer(s) B1 or instead thereof, the agents I may also contain copolymers B2 which comprise as additional structural units structural units of formula (B-II), in which n denotes the number 3.

Further more preferred agents are characterized in that they contain as copolymer B a copolymer B2 which
contains a structural unit according to formula (B-I), in which R denotes a methyl group and X denotes methosulfate,
contains further structural units according to formula (B-II), in which n denotes 1 methylene unit
contains further structural units according to formula (B-II), in which n denotes 3 methylene units.

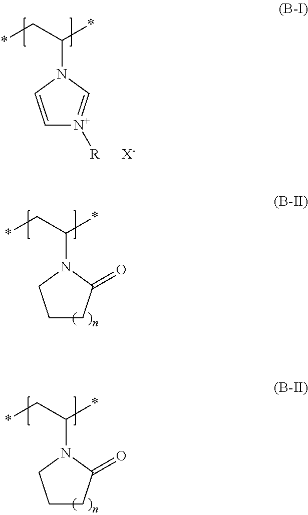

In this case too, it is more preferred for the copolymers B2 to contain, in addition to polymer units which arise from the incorporation of the stated structural units according to formula (B-I) and (B-II) into the copolymer, at most about 5 wt. %, preferably at most about 1 wt. %, of polymer units originating from the incorporation of other monomers. The copolymers B2 are preferably exclusively synthesized from structural units of the formulae (B-I) and (B-II) and may be described by the general formula

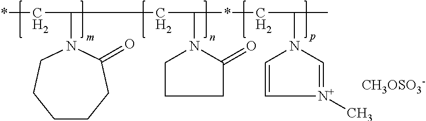

wherein the indices m, n and p vary depending on the molar mass of the polymer and are not intended to mean that the copolymers are block copolymers. Instead, structural units of the formula (B-I) and the formula (B-II) may be present randomly distributed in the molecule.

In combination with the copolymers A, copolymers B which have proven particularly effective are those in which copolymer B is formed from
3-methyl-1-vinylimidazolium methylsulfate (B1),
N-vinylpyrrolidone (B2a),
N-vinylcaprolactam (B2b).

With regard to the cosmetic properties of the agents contemplated herein, it has proven advantageous for copolymer B to consist in a proportion of at least about 70 wt. %, preferably at least about 80 wt. %, preferably at least about 90 wt. % and in particular at least about 95 wt. % of the structural units (B1), (B2) and (B3).

Such N-methylvinylimidazole/vinylpyrrolidone/vinylcaprolactam copolymers are designated POLYQUATERNIUM-46 according to INCI nomenclature and are obtainable for example from BASF under the trade name Luviquat® Hold.

Particularly preferred copolymers B2 contain about 1 to about 20 mol %, preferably about 5 to about 15 mol % and in particular about 10 mol % of structural units according to formula (B-I) and about 30 to about 50 mol %, preferably about 35 to about 45 mol % and in particular about 40 mol % of structural units according to formula (B-II) with n=1 and about 40 to about 60 mol %, preferably about 45 to about 55 mol % and in particular about 60 mol % of structural units according to formula (B-II) with n=3.

More preferred agents contain a copolymer B2 which has molar masses within a specific range. Preferred hair deforming agents are here those in which copolymer B2 has a molar mass of about 100 to about 1000 kDa, preferably of about 250 to about 900 kDa, more preferably of about 500 to about 850 kDa and in particular of about 650 to about 710 kDa.

In addition to copolymer(s) B1 and/or B2 or instead thereof, the agents may also contain copolymers B3, which comprise as additional structural units structural units of formula (B-II), in which n denotes the number 3 and further distructural units from the group of vinylimidazole units and further structural units from the group of acrylamide and/or methacrylamide units.

Particularly preferred agents are characterized in that they contain as copolymer B a copolymer B3 which
contains a structural unit according to formula (B-I), in which R denotes a methyl group and X denotes methosulfate,
contains further structural units according to formula (B-II), in which n denotes 1 methylene unit
contains further structural units according to formula (B-III)
contains further structural units according to formula (B-IV)

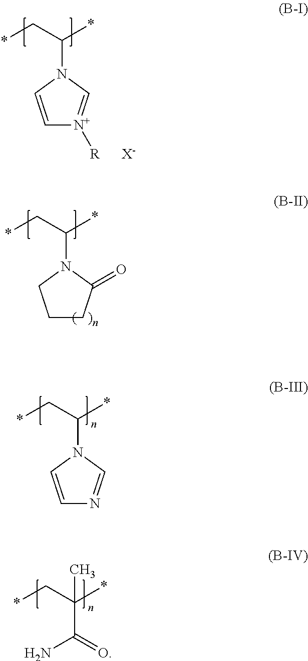

In this case too, it is more preferred for the copolymers B3 to contain, in addition to polymer units which arise from the incorporation of the stated structural units according to formula (B-I), (B-II), (B-III) and (B-IV) into the copolymer, at most about 5 wt. %, preferably at most about 1 wt. %, of polymer units originating from the incorporation of other monomers. The copolymers B3 are preferably exclusively synthesized from structural units of formulae (B-I), (B-II), (B-III) and (B-IV) and may be described by the general formula

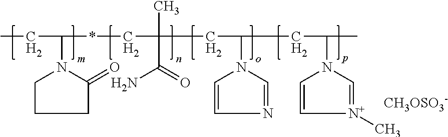

wherein the indices m, n, o and p vary depending on the molar mass of the polymer and are not intended to mean that the copolymers are block copolymers. Instead structural units of formulae (B-I), (B-II), (B-III) and (B-IV) may be present randomly distributed in the molecule.

Such N-methylvinylimidazole/vinylpyrrolidone/vinylcaprolactam copolymers are designated POLYQUATERNIUM-68 according to INCI nomenclature and are obtainable for example from BASF under the trade name Luviquat® Supreme.

Particularly preferred copolymers B3 contain about 1 to about 12 mol %, preferably about 3 to about 9 mol % and in particular about 6 mol % of structural units according to formula (B-I) and about 45 to about 65 mol %, preferably about 50 to about 60 mol % and in particular about 55 mol % of structural units according to formula (B-II) with n=1 and about 1 to about 20 mol %, preferably about 5 to about 15 mol % and in particular about 10 mol % of structural units according to formula (B-III) and about 20 to about 40 mol %, preferably about 25 to about 35 mol % and in particular about 29 mol % of structural units according to formula (B-IV).

More preferred agents contain a copolymer B3 which has molar masses within a specific range. Preferred hair deforming agents are here those in which copolymer B3 has a molar mass of about 100 to about 500 kDa, preferably of about 150 to about 400 kDa, more preferably of about 250 to about 350 kDa and in particular of about 290 to about 310 kDa.

The proportion by weight of copolymer B in the total weight of cosmetic agents contemplated herein preferably amounts to about 0.05 to about 10 wt. %, preferably about 0.1 to about 7.0 wt. % and in particular about 0.2 to about 5.0 wt. %.

The cosmetic agents contemplated herein are distinguished from cosmetic agents with alternative vinylpyrrolidone copolymers not only by the above-stated advantages but in particular also by an improved level of hold. For the cosmetic properties of the agents, a weight ratio of polymers A and B in the cosmetic agent of between about 8:1 and about 1:8, preferably between about 6:1 and about 1:6 and in particular between about 4:1 and about 1:4 has proven particularly advantageous. It has here additionally proven advantageous to use one of the two copolymers in excess. Preferred cosmetic agents are therefore those in which the weight ratio of copolymer A to copolymer B amounts to more than about 1:2, preferably more than about 2:5, preferably more than about 1:3 and in particular more than about 1:4. More preferred cosmetic agents are those in which the weight ratio of copolymer A to copolymer B is about 1:2 to about 1:20, preferably about 2:5 to about 1:15, preferably about 1:3 to about 1:12 and in particular about 1:4 to about 1:10.

Copolymer A is used in the cosmetic agents preferably in partially neutralized or neutralized form. At least one organic acid is preferably used for neutralization. More preferred organic acids are carboxylic acids, in particular short-chain alkanoic acids and hydroxycarboxylic acids. The use of formic acid, acetic acid and lactic acid, in particular of lactic acid, has proven particularly advantageous. The acid(s) is/are used in the agents preferably in a quantity which does not exceed the quantity needed to neutralize copolymer A. The quantities of carboxylic acid, in particular lactic acid, used in the agents preferably amount to about 80 to about 100%, more preferably about 90 to about 100% and in particular about 95 to about 100% of the quantity required for complete neutralization of the copolymer A. In a preferred embodiment, the proportion by weight of carboxylic acid, in particular of lactic acid in the total weight of the cosmetic agent amounts to about 0.1 to about 4.0 wt. %, preferably about 0.2 to about 3.0 wt. % and in particular about 0.5 to about 2.0 wt. %.

In addition to the previously described copolymers and carrier substances, the cosmetic agents contemplated herein may contain further ingredients. The group of these further ingredients in particular includes cosmetically active auxiliary substances and additives.

The cosmetic agents contain as preferred component at least one quaternary ammonium compound. Monomeric or polymeric active substances may be used as the quaternary ammonium compound.

From the plurality of possible monomeric quaternary ammonium compounds, the compounds from the groups:
trimethylalkylammonium halides;
ester quats
quaternary imidazolines have proven particularly effective.

The group of trimethylalkylammonium halides in particular includes the compounds of formula (Tkat1).

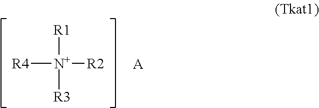

In the formula (Tkat1), R1, R2, R3 and R4 in each case mutually independently denote hydrogen, a methyl group, a phenyl group, a benzyl group, a saturated, branched or unbranched alkyl residue with a chain length of 8 to 30 carbon atoms, which may optionally be substituted with one or more hydroxyl groups. A denotes a physiologically acceptable anion, for example halides such as chloride or bromide and methosulfates.

Examples of compounds of formula (Tkat1) are lauryltrimethylammonium chloride, cetyltrimethylammonium chloride, cetyltrimethylammonium bromide, cetyltrimethylammonium methosulfate, dicetyldimethylammonium chloride, tricetylmethylammonium chloride, stearyltrimethylammonium chloride, distearyldimethylammonium chloride, lauryldimethylbenzylammonium chloride, behenyltrimethylammonium chloride, behenyltrimethylammonium bromide and behenyltrimethylammonium methosulfate. Preferred cosmetic agents contain a monomeric quaternary ammonium compound from the group of trimethylalkylammonium halides.

Further quaternary ammonium compounds which are more preferred are the cationic betaine esters of formula (Tkat1-2.1).

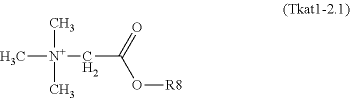

More preferred ester quats are those with the trade names Armocare VGH-70, and Dehyquart® F-75, Dehyquart® L80, Stepantex® VS 90 and Akypoquat® 131.

A further group are quaternary imidazoline compounds. The formula (Tkat2) illustrated below shows the structure of these compounds.

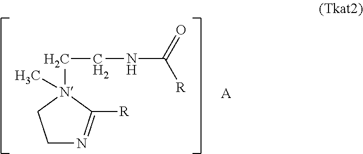

The residues R mutually independently in each case denote a saturated or unsaturated, linear or branched hydrocarbon residue with a chain length of 8 to 30 carbon atoms. The preferred compounds of the formula (Tkat2) in each case contain the identical hydrocarbon residue for R. The chain length of the residues R preferably amounts to 12 to 21 carbon atoms. A denotes an anion as previously described. Examples which are particularly preferred are obtainable for example under NCI names Quaternium-27, Quaternium-72, Quaternium-83, Quaternium-87 and Quaternium-91. Quaternium-91 is most preferred.

With regard to cosmetic action, advantageous cosmetic agents have proven to be those in which the proportion by weight of the monomeric quaternary ammonium compound in the total weight of the agent amounts to about 0.05 to about 3.0 wt. %, preferably about 0.1 to about 2.0 wt. % and in particular about 0.2 to about 1.0 wt. %.

Suitable auxiliary substances and additives which may be mentioned are in particular additional conditioning substances.

The agent may for example contain at least one protein hydrolysate and/or one of the derivatives thereof as a conditioning substance of another compound class. Protein hydrolysates are product mixtures which are obtained by acidically, basically or enzymatically catalyzed degradation of proteins. As used herein, the term protein hydrolysates also covers total hydrolysates and individual amino acids and the derivatives thereof and mixtures of different amino acids. The molecular weight of the protein hydrolysates which may be used herein is between about 75, the molecular weight of glycine, and about 200,000, the molecular weight preferably amounting to about 75 to about 50,000 and particularly preferably to about 75 to about 20,000 daltons.

The agent contemplated may furthermore contain at least one vitamin, a provitamin, a vitamin precursor and/or one of the derivatives thereof as a conditioning substance. Preferred vitamins, provitamins and vitamin precursors useful herein are those which are conventionally assigned to groups A, B, C, E, F and H.

Like the addition of glycerol and/or propylene glycol, the addition of panthenol increases the flexibility of the polymer film formed using the agent.

The agents contemplated herein may furthermore contain at least one plant extract, but also mono- or oligosaccharides and/or lipids as conditioning substance.

Oil bodies are furthermore suitable as a conditioning substance. Natural and synthetic cosmetic oil bodies include, for example, vegetable oils, liquid paraffin oils, isoparaffin oils and synthetic hydrocarbons and di-n-alkyl ethers having a total of between 12 to 36 C atoms, in particular 12 to 24 C atoms. Preferred cosmetic agents contain at least one oil body, preferably at least one oil body from the group of silicone oils. The group of silicone oils in particular includes dimethicones, which also include cyclomethicones, amino-functional silicones and dimethiconols. The dimethicones may be both linear and branched and cyclic or cyclic and branched. Suitable silicone oils or silicone gums are in particular dialkyl- and alkylarylsiloxanes, such as for example dimethylpolysiloxane and methylphenylpolysiloxane, and the alkoxylated, quaternized or also anionic derivatives thereof. Preference is given to cyclic and linear polydialkylsiloxanes, the alkoxylated and/or aminated derivatives thereof, dihydroxypolydimethylsiloxanes and polyphenylalkylsiloxanes.

Ester oils, i.e. esters of C₆-C₃₀ fatty acids with C₂-C₃₀ fatty alcohols, preferably monoesters of the fatty acids with alcohols with 2 to 24 C atoms such as for example isopropyl myristate (Rilanit® IPM), isononanoic acid C₁₆₋₁₈ alkyl esters (Cetiol® SN), 2-ethylhexyl palmitate (Cegesoft® 24), stearic acid 2-ethylhexyl ester, (Cetiol® 868), cetyl oleate, glycerol tricaprylate, coco fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are further preferred conditioning oil bodies.

Additional suitable conditioning substances are dicarboxylic acid esters, symmetrical, asymmetrical or cyclic esters of carbonic acid with fatty alcohols, trifatty acid esters of saturated and/or unsaturated linear and/or branched fatty acids with glycerol, or fatty acid partial glycerides, which should be understood to mean monoglycerides, diglycerides and the technical mixtures thereof.

With regard to cosmetic action, advantageous cosmetic agents have proven to be those in which the proportion by weight of the oil body in the total weight of the agent amounts to about 0.01 to about 5.0 wt. %, preferably about 0.02 to about 4.0 wt. % and in particular about 0.05 to about 2.0 wt. %.

The following tables show the composition of some preferred cosmetic agents (details in wt. % relative to the total weight of the cosmetic agent unless otherwise stated).

| | Formula 1 | Formula 2 | Formula 3 | Formula 4 | Formula 5 |
|---|---|---|---|---|---|
| Copolymer A | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Water, misc. | Ad 100 | Ad 100 | Ad 100 | Ad 100 | Ad 100 |
| | Formula 6 | Formula 7 | Formula 8 | Formula 9 | Formula 10 |
| Copolymer A¹⁾ | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Water, misc. | Ad 100 | Ad 100 | Ad 100 | Ad 100 | Ad 100 |
| | Formula 11 | Formula 12 | Formula 13 | Formula 14 | Formula 15 |
| Copolymer A | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Water, misc. | Ad 100 | Ad 100 | Ad 100 | Ad 100 | Ad 100 |
| | Formula 16 | Formula 17 | Formula 18 | Formula 19 | Formula 20 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Water, misc. | Ad 100 | Ad 100 | Ad 100 | Ad 100 | Ad 100 |
| | Formula 21 | Formula 22 | Formula 23 | Formula 24 | Formula 25 |
| Copolymer A | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Carboxylic acid³⁾ | 80 to 100 | 90 to 100 | 95 to 100 | 98 | 98 |
| Water, misc. | Ad 100 | Ad 100 | Ad 100 | Ad 100 | Ad 100 |
| | Formula 26 | Formula 27 | Formula 28 | Formula 29 | Formula 30 |
| Copolymer A¹⁾ | 0.05 to 10 | 0.1 to 7.0 | 0.2 to 5.0 | 0.4 | 1.2 |
| Copolymer B | 0.05 to 10 | 0.1 to 7.0 | 0.2 to 5.0 | 0.8 | 0.8 |
| Carboxylic acid³⁾ | 80 to 100 | 90 to 100 | 95 to 100 | 98 | 98 |
| Water, misc. | Ad 100 | Ad 100 | Ad 100 | Ad 100 | Ad 100 |
| | Formula 31 | Formula 32 | Formula 33 | Formula 34 | Formula 35 |
| Copolymer A | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Carboxylic acid³⁾ | 80 to 100 | 90 to 100 | 95 to 100 | 98 | 98 |
| Water, misc. | Ad 100 | Ad 100 | Ad 100 | Ad 100 | Ad 100 |
| | Formula 36 | Formula 37 | Formula 38 | Formula 39 | Formula 40 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Carboxylic acid³⁾ | 80 to 100 | 90 to 100 | 95 to 100 | 98 | 98 |
| Water, misc. | Ad 100 | Ad 100 | Ad 100 | Ad 100 | Ad 100 |
| | Formula 41 | Formula 42 | Formula 43 | Formula 44 | Formula 45 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Lactic acid³⁾ | 80 to 100 | 90 to 100 | 95 to 100 | 98 | 98 |
| Water, misc. | Ad 100 | Ad 100 | Ad 100 | Ad 100 | Ad 100 |
| | Formula 46 | Formula 47 | Formula 48 | Formula 49 | Formula 50 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Water, misc. | Ad 100 | Ad 100 | Ad 100 | Ad 100 | Ad 100 |
| | Formula 51 | Formula 52 | Formula 53 | Formula 54 | Formula 55 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Oil body | 0.01 to 5.0 | 0.02 to 4.0 | 0.05 to 2.0 | 0.1 | 0.1 |
| Water, misc. | Ad 100 | Ad 100 | Ad 100 | Ad 100 | Ad 100 |
| | Formula 56 | Formula 57 | Formula 58 | Formula 59 | Formula 60 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Water | 40 to 99 | 50 to 98 | 60 to 95 | 97 | 92 |
| Misc. | Ad 100 | Ad 100 | Ad 100 | Ad 100 | Ad 100 |
| | Formula 61 | Formula 62 | Formula 63 | Formula 64 | Formula 65 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Alkyltrimethyl- | 0.05 to 3.0 | 0.1 to 2.0 | 0.2 to 1.0 | 0.5 | 0.3 |
| ammonium | | | | | |
| chloride | | | | | |
| Water | 40 to 99 | 50 to 98 | 60 to 95 | 97 | 92 |
| Misc. | Ad 100 | Ad 100 | Ad 100 | Ad 100 | Ad 100 |
| | Formula 66 | Formula 67 | Formula 68 | Formula 69 | Formula 70 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Oil body | 0.01 to 5.0 | 0.02 to 4.0 | 0.05 to 2.0 | 0.1 | 0.1 |
| Water | 40 to 99 | 50 to 98 | 60 to 95 | 97 | 92 |
| Misc. | Ad 100 | Ad 100 | Ad 100 | Ad 100 | Ad 100 |
| | Formula 71 | Formula 72 | Formula 73 | Formula 74 | Formula 75 |
| Copolymer A¹⁾ | 0.05 to 10 | 0.1 to 7.0 | 0.2 to 5.0 | 0.4 | 1.2 |
| Copolymer B²⁾ | 0.05 to 10 | 0.1 to 7.0 | 0.2 to 5.0 | 0.8 | 0.8 |
| Lactic acid³⁾ | 80 to 100 | 90 to 100 | 95 to 100 | 98 | 98 |
| Oil body | 0.01 to 5.0 | 0.02 to 4.0 | 0.05 to 2.0 | 0.1 | 0.1 |
| Water | 40 to 99 | 50 to 98 | 60 to 95 | 97 | 92 |
| Misc. | Ad 100 | Ad 100 | Ad 100 | Ad 100 | Ad 100 |

¹⁾Copolymers A, formed from at least one monomer (A1) from the group acrylic acid, methacrylic acid, C₁-C₆ alkyl acrylic acid esters, C₁-C₆ alkyl methacrylic acid esters, at least one monomer (A2) from the group acrylamide, methacrylamide, mono-(C₁-C₄)-alkylamino-(C₁-C₄)-alkyl acrylate, di-(C₁-C₄)-alkylamino-(C₁-C₄)-alkyl acrylate, mono-(C₁-C₄)-alkylamino-(C₁-C₄)- alkyl methacrylate, di-(C₁-C₄)-alkylamino-(C₁-C₄)-alkyl methacrylate, at least one monomer (A3) from the group of C₁₀₋₃₀ alkyl PEG-20 itaconates, wherein at least about 95 wt. % of the copolymer A consists of the structural units (A1), (A2) and (A3).
²⁾Copolymer B is formed from 3-methyl-1-vinylimidazolium methylsulfate (B1), N-vinylpyrrolidone (B2a), N-vinylcaprolactam (B2b), wherein at least about 95 wt. % of the copolymer B consists of the structural units (B1), (B2) and (B3).
³⁾% of the quantity required for complete neutralization of copolymer A.

The cosmetic agents contemplated herein may be formulated in any forms conventional for cosmetic agents, for example in the form of solutions, which may for example be applied onto the hair as a hair lotion, in the form of creams, emulsions, waxes, gels or also surfactant-containing foaming solutions or other preparations which are suitable for use on the hair. In an alternative embodiment, these agents may however also assume gel or cream form, wherein transparent gels are more preferred.

The compositions contemplated herein are in particular highly suitable for stabilizing gas bubbles in the agent. In this way, air or other gases or gas mixtures can be readily incorporated into the agents in such a manner as to be stable in the long-term. This may optionally proceed during production of the agents, by exposing the agent to gas, preferably air, before packaging and packaging a product containing visible gas bubbles. The agents preferably assume the form of a foam. A foam is here a preparation comprising gas filled bubbles surrounded by liquid (liquid foam) or solid (stiff foam) walls. The compositions listed in the following table are preferably stiff foams. The density of preferred compositions amounts to about 0.3 to about 1.0 g/cm³, preferably about 0.4 to about 0.9 g/cm³ and in particular about 0.5 to about 0.8 g/cm³. Such foams may for example be produced by beating the preparation in a suitable mixer or by exposure to a suitable gas, preferably air.

The following tables show the composition of some preferred cosmetic foams. In this table the left-hand column (“formula x”) refers in each case to one of the exemplary cosmetic compositions listed in the tables disclosed further above. The further columns two to seven (“density”) in each case indicate the density of the corresponding cosmetic composition.

In other words, a cosmetic preparation according to line 12, column 5 of the following table comprises a cosmetic agent according to formula 11 with a density of 0.44 g/cm³.

| | Density [g/cm³] | | | | | |
|---|---|---|---|---|---|---|
| Formula 1 | 0.3 to 1.0 | 0.93 | 0.4 to 0.9 | 0.44 | 0.5 to 0.8 | 0.61 |
| Formula 2 | 0.3 to 1.0 | 0.93 | 0.4 to 0.9 | 0.44 | 0.5 to 0.8 | 0.61 |
| Formula 3 | 0.3 to 1.0 | 0.93 | 0.4 to 0.9 | 0.44 | 0.5 to 0.8 | 0.61 |
| Formula 4 | 0.3 to 1.0 | 0.93 | 0.4 to 0.9 | 0.44 | 0.5 to 0.8 | 0.61 |
| Formula 5 | 0.3 to 1.0 | 0.93 | 0.4 to 0.9 | 0.44 | 0.5 to 0.8 | 0.61 |
| Formula 6 | 0.3 to 1.0 | 0.93 | 0.4 to 0.9 | 0.44 | 0.5 to 0.8 | 0.61 |
| Formula 7 | 0.3 to 1.0 | 0.93 | 0.4 to 0.9 | 0.44 | 0.5 to 0.8 | 0.61 |
| Formula 8 | 0.3 to 1.0 | 0.93 | 0.4 to 0.9 | 0.44 | 0.5 to 0.8 | 0.61 |
| Formula 9 | 0.3 to 1.0 | 0.93 | 0.4 to 0.9 | 0.44 | 0.5 to 0.8 | 0.61 |
| Formula 10 | 0.3 to 1.0 | 0.93 | 0.4 to 0.9 | 0.44 | 0.5 to 0.8 | 0.61 |
| Formula 11 | 0.3 to 1.0 | 0.93 | 0.4 to 0.9 | 0.44 | 0.5 to 0.8 | 0.61 |
| Formula 12 | 0.3 to 1.0 | 0.93 | 0.4 to 0.9 | 0.44 | 0.5 to 0.8 | 0.61 |
| Formula 13 | 0.3 to 1.0 | 0.93 | 0.4 to 0.9 | 0.44 | 0.5 to 0.8 | 0.61 |
| Formula 14 | 0.3 to 1.0 | 0.93 | 0.4 to 0.9 | 0.44 | 0.5 to 0.8 | 0.61 |
| Formula 15 | 0.3 to 1.0 | 0.93 | 0.4 to 0.9 | 0.44 | 0.5 to 0.8 | 0.61 |
| Formula 16 | 0.3 to 1.0 | 0.93 | 0.4 to 0.9 | 0.44 | 0.5 to 0.8 | 0.61 |
| Formula 17 | 0.3 to 1.0 | 0.93 | 0.4 to 0.9 | 0.44 | 0.5 to 0.8 | 0.61 |
| Formula 18 | 0.3 to 1.0 | 0.93 | 0.4 to 0.9 | 0.44 | 0.5 to 0.8 | 0.61 |
| Formula 19 | 0.3 to 1.0 | 0.93 | 0.4 to 0.9 | 0.44 | 0.5 to 0.8 | 0.61 |
| Formula 20 | 0.3 to 1.0 | 0.93 | 0.4 to 0.9 | 0.44 | 0.5 to 0.8 | 0.61 |
| Formula 21 | 0.3 to 1.0 | 0.93 | 0.4 to 0.9 | 0.44 | 0.5 to 0.8 | 0.61 |
| Formula 22 | 0.3 to 1.0 | 0.93 | 0.4 to 0.9 | 0.44 | 0.5 to 0.8 | 0.61 |
| Formula 23 | 0.3 to 1.0 | 0.93 | 0.4 to 0.9 | 0.44 | 0.5 to 0.8 | 0.61 |
| Formula 24 | 0.3 to 1.0 | 0.93 | 0.4 to 0.9 | 0.44 | 0.5 to 0.8 | 0.61 |
| Formula 25 | 0.3 to 1.0 | 0.93 | 0.4 to 0.9 | 0.44 | 0.5 to 0.8 | 0.61 |
| Formula 26 | 0.3 to 1.0 | 0.93 | 0.4 to 0.9 | 0.44 | 0.5 to 0.8 | 0.61 |
| Formula 27 | 0.3 to 1.0 | 0.93 | 0.4 to 0.9 | 0.44 | 0.5 to 0.8 | 0.61 |
| Formula 28 | 0.3 to 1.0 | 0.93 | 0.4 to 0.9 | 0.44 | 0.5 to 0.8 | 0.61 |
| Formula 29 | 0.3 to 1.0 | 0.93 | 0.4 to 0.9 | 0.44 | 0.5 to 0.8 | 0.61 |
| Formula 30 | 0.3 to 1.0 | 0.93 | 0.4 to 0.9 | 0.44 | 0.5 to 0.8 | 0.61 |
| Formula 31 | 0.3 to 1.0 | 0.93 | 0.4 to 0.9 | 0.44 | 0.5 to 0.8 | 0.61 |
| Formula 32 | 0.3 to 1.0 | 0.93 | 0.4 to 0.9 | 0.44 | 0.5 to 0.8 | 0.61 |
| Formula 33 | 0.3 to 1.0 | 0.93 | 0.4 to 0.9 | 0.44 | 0.5 to 0.8 | 0.61 |
| Formula 34 | 0.3 to 1.0 | 0.93 | 0.4 to 0.9 | 0.44 | 0.5 to 0.8 | 0.61 |
| Formula 35 | 0.3 to 1.0 | 0.93 | 0.4 to 0.9 | 0.44 | 0.5 to 0.8 | 0.61 |
| Formula 36 | 0.3 to 1.0 | 0.93 | 0.4 to 0.9 | 0.44 | 0.5 to 0.8 | 0.61 |
| Formula 37 | 0.3 to 1.0 | 0.93 | 0.4 to 0.9 | 0.44 | 0.5 to 0.8 | 0.61 |
| Formula 38 | 0.3 to 1.0 | 0.93 | 0.4 to 0.9 | 0.44 | 0.5 to 0.8 | 0.61 |
| Formula 39 | 0.3 to 1.0 | 0.93 | 0.4 to 0.9 | 0.44 | 0.5 to 0.8 | 0.61 |
| Formula 40 | 0.3 to 1.0 | 0.93 | 0.4 to 0.9 | 0.44 | 0.5 to 0.8 | 0.61 |
| Formula 41 | 0.3 to 1.0 | 0.93 | 0.4 to 0.9 | 0.44 | 0.5 to 0.8 | 0.61 |
| Formula 42 | 0.3 to 1.0 | 0.93 | 0.4 to 0.9 | 0.44 | 0.5 to 0.8 | 0.61 |
| Formula 43 | 0.3 to 1.0 | 0.93 | 0.4 to 0.9 | 0.44 | 0.5 to 0.8 | 0.61 |
| Formula 44 | 0.3 to 1.0 | 0.93 | 0.4 to 0.9 | 0.44 | 0.5 to 0.8 | 0.61 |
| Formula 45 | 0.3 to 1.0 | 0.93 | 0.4 to 0.9 | 0.44 | 0.5 to 0.8 | 0.61 |
| Formula 46 | 0.3 to 1.0 | 0.93 | 0.4 to 0.9 | 0.44 | 0.5 to 0.8 | 0.61 |
| Formula 47 | 0.3 to 1.0 | 0.93 | 0.4 to 0.9 | 0.44 | 0.5 to 0.8 | 0.61 |
| Formula 48 | 0.3 to 1.0 | 0.93 | 0.4 to 0.9 | 0.44 | 0.5 to 0.8 | 0.61 |
| Formula 49 | 0.3 to 1.0 | 0.93 | 0.4 to 0.9 | 0.44 | 0.5 to 0.8 | 0.61 |
| Formula 50 | 0.3 to 1.0 | 0.93 | 0.4 to 0.9 | 0.44 | 0.5 to 0.8 | 0.61 |
| Formula 51 | 0.3 to 1.0 | 0.93 | 0.4 to 0.9 | 0.44 | 0.5 to 0.8 | 0.61 |
| Formula 52 | 0.3 to 1.0 | 0.93 | 0.4 to 0.9 | 0.44 | 0.5 to 0.8 | 0.61 |
| Formula 53 | 0.3 to 1.0 | 0.93 | 0.4 to 0.9 | 0.44 | 0.5 to 0.8 | 0.61 |
| Formula 54 | 0.3 to 1.0 | 0.93 | 0.4 to 0.9 | 0.44 | 0.5 to 0.8 | 0.61 |
| Formula 55 | 0.3 to 1.0 | 0.93 | 0.4 to 0.9 | 0.44 | 0.5 to 0.8 | 0.61 |
| Formula 56 | 0.3 to 1.0 | 0.93 | 0.4 to 0.9 | 0.44 | 0.5 to 0.8 | 0.61 |
| Formula 57 | 0.3 to 1.0 | 0.93 | 0.4 to 0.9 | 0.44 | 0.5 to 0.8 | 0.61 |
| Formula 58 | 0.3 to 1.0 | 0.93 | 0.4 to 0.9 | 0.44 | 0.5 to 0.8 | 0.61 |
| Formula 59 | 0.3 to 1.0 | 0.93 | 0.4 to 0.9 | 0.44 | 0.5 to 0.8 | 0.61 |
| Formula 60 | 0.3 to 1.0 | 0.93 | 0.4 to 0.9 | 0.44 | 0.5 to 0.8 | 0.61 |
| Formula 61 | 0.3 to 1.0 | 0.93 | 0.4 to 0.9 | 0.44 | 0.5 to 0.8 | 0.61 |
| Formula 62 | 0.3 to 1.0 | 0.93 | 0.4 to 0.9 | 0.44 | 0.5 to 0.8 | 0.61 |
| Formula 63 | 0.3 to 1.0 | 0.93 | 0.4 to 0.9 | 0.44 | 0.5 to 0.8 | 0.61 |
| Formula 64 | 0.3 to 1.0 | 0.93 | 0.4 to 0.9 | 0.44 | 0.5 to 0.8 | 0.61 |
| Formula 65 | 0.3 to 1.0 | 0.93 | 0.4 to 0.9 | 0.44 | 0.5 to 0.8 | 0.61 |
| Formula 66 | 0.3 to 1.0 | 0.93 | 0.4 to 0.9 | 0.44 | 0.5 to 0.8 | 0.61 |
| Formula 67 | 0.3 to 1.0 | 0.93 | 0.4 to 0.9 | 0.44 | 0.5 to 0.8 | 0.61 |
| Formula 68 | 0.3 to 1.0 | 0.93 | 0.4 to 0.9 | 0.44 | 0.5 to 0.8 | 0.61 |
| Formula 69 | 0.3 to 1.0 | 0.93 | 0.4 to 0.9 | 0.44 | 0.5 to 0.8 | 0.61 |
| Formula 70 | 0.3 to 1.0 | 0.93 | 0.4 to 0.9 | 0.44 | 0.5 to 0.8 | 0.61 |
| Formula 71 | 0.3 to 1.0 | 0.93 | 0.4 to 0.9 | 0.44 | 0.5 to 0.8 | 0.61 |
| Formula 72 | 0.3 to 1.0 | 0.93 | 0.4 to 0.9 | 0.44 | 0.5 to 0.8 | 0.61 |
| Formula 73 | 0.3 to 1.0 | 0.93 | 0.4 to 0.9 | 0.44 | 0.5 to 0.8 | 0.61 |
| Formula 74 | 0.3 to 1.0 | 0.93 | 0.4 to 0.9 | 0.44 | 0.5 to 0.8 | 0.61 |
| Formula 75 | 0.3 to 1.0 | 0.93 | 0.4 to 0.9 | 0.44 | 0.5 to 0.8 | 0.61 |

In an alternative embodiment, the cosmetic agents contemplated herein may be packaged as pump or aerosol sprays. In addition to further active and auxiliary substances, preferred aerosol sprays contain a propellant. Propellants (propellant gases) suitable for use herein are propane, n-butane, iso-butane, dimethyl ether (DME), nitrogen, air, nitrous oxide, 1,1-difluoroethane, specifically both individually and in combination. Hydrophilic propellant gases, such as for example carbon dioxide, may advantageously be used for the purposes herein if a small proportion of hydrophilic gases is selected and a lipophilic propellant gas (for example propane/butane) is present in excess. Dimethyl ether, propane, n-butane, iso-butane and mixtures of these propellant gases are more preferred. The use of propane/butane mixtures or isobutane is particularly preferred. Cosmetic agents which, relative to the total weight thereof, contain the propellant in a quantity of about 2.0 to about 20 wt. %, preferably about 4.0 to about 15 wt. % and more preferably about 5.0 to about 10 wt. %, are preferred.

The following tables show the composition of some preferred propellant-containing cosmetic agents. In this table the left-hand column (“formula x”) refers in each case to one of the exemplary cosmetic compositions listed in the tables disclosed further above. The further columns two to seven (“propellant”) in each case indicate the quantity of propellant added to the corresponding cosmetic composition. These indications in “wt. %” relate to the total weight of the cosmetic composition of the respective “formula x” without propellant.

In other words, a cosmetic preparation according to line 12, column 5 of the following table comprises a 20:1 mixture of the propellant-free cosmetic agent according to formula 11 with a propane/butane mixture.

| | Propellant [wt. %] | | | | | |
|---|---|---|---|---|---|---|
| Formula 1 | 2.0 to 10 | 5.0 | 2.0 to 10 P/B* | 5.0 P/B | 2.0 to 10 iB** | 5.0 iB |
| Formula 2 | 2.0 to 10 | 5.0 | 2.0 to 10 P/B* | 5.0 P/B | 2.0 to 10 iB** | 5.0 iB |
| Formula 3 | 2.0 to 10 | 5.0 | 2.0 to 10 P/B* | 5.0 P/B | 2.0 to 10 iB** | 5.0 iB |
| Formula 4 | 2.0 to 10 | 5.0 | 2.0 to 10 P/B* | 5.0 P/B | 2.0 to 10 iB** | 5.0 iB |
| Formula 5 | 2.0 to 10 | 5.0 | 2.0 to 10 P/B* | 5.0 P/B | 2.0 to 10 iB** | 5.0 iB |
| Formula 6 | 2.0 to 10 | 5.0 | 2.0 to 10 P/B* | 5.0 P/B | 2.0 to 10 iB** | 5.0 iB |
| Formula 7 | 2.0 to 10 | 5.0 | 2.0 to 10 P/B* | 5.0 P/B | 2.0 to 10 iB** | 5.0 iB |
| Formula 8 | 2.0 to 10 | 5.0 | 2.0 to 10 P/B* | 5.0 P/B | 2.0 to 10 iB** | 5.0 iB |
| Formula 9 | 2.0 to 10 | 5.0 | 2.0 to 10 P/B* | 5.0 P/B | 2.0 to 10 iB** | 5.0 iB |
| Formula 10 | 2.0 to 10 | 5.0 | 2.0 to 10 P/B* | 5.0 P/B | 2.0 to 10 iB** | 5.0 iB |
| Formula 11 | 2.0 to 10 | 5.0 | 2.0 to 10 P/B* | 5.0 P/B | 2.0 to 10 iB** | 5.0 iB |
| Formula 12 | 2.0 to 10 | 5.0 | 2.0 to 10 P/B* | 5.0 P/B | 2.0 to 10 iB** | 5.0 iB |
| Formula 13 | 2.0 to 10 | 5.0 | 2.0 to 10 P/B* | 5.0 P/B | 2.0 to 10 iB** | 5.0 iB |
| Formula 14 | 2.0 to 10 | 5.0 | 2.0 to 10 P/B* | 5.0 P/B | 2.0 to 10 iB** | 5.0 iB |
| Formula 15 | 2.0 to 10 | 5.0 | 2.0 to 10 P/B* | 5.0 P/B | 2.0 to 10 iB** | 5.0 iB |
| Formula 16 | 2.0 to 10 | 5.0 | 2.0 to 10 P/B* | 5.0 P/B | 2.0 to 10 iB** | 5.0 iB |
| Formula 17 | 2.0 to 10 | 5.0 | 2.0 to 10 P/B* | 5.0 P/B | 2.0 to 10 iB** | 5.0 iB |
| Formula 18 | 2.0 to 10 | 5.0 | 2.0 to 10 P/B* | 5.0 P/B | 2.0 to 10 iB** | 5.0 iB |
| Formula 19 | 2.0 to 10 | 5.0 | 2.0 to 10 P/B* | 5.0 P/B | 2.0 to 10 iB** | 5.0 iB |
| Formula 20 | 2.0 to 10 | 5.0 | 2.0 to 10 P/B* | 5.0 P/B | 2.0 to 10 iB** | 5.0 iB |
| Formula 21 | 2.0 to 10 | 5.0 | 2.0 to 10 P/B* | 5.0 P/B | 2.0 to 10 iB** | 5.0 iB |
| Formula 22 | 2.0 to 10 | 5.0 | 2.0 to 10 P/B* | 5.0 P/B | 2.0 to 10 iB** | 5.0 iB |
| Formula 23 | 2.0 to 10 | 5.0 | 2.0 to 10 P/B* | 5.0 P/B | 2.0 to 10 iB** | 5.0 iB |
| Formula 24 | 2.0 to 10 | 5.0 | 2.0 to 10 P/B* | 5.0 P/B | 2.0 to 10 iB** | 5.0 iB |
| Formula 25 | 2.0 to 10 | 5.0 | 2.0 to 10 P/B* | 5.0 P/B | 2.0 to 10 iB** | 5.0 iB |
| Formula 26 | 2.0 to 10 | 5.0 | 2.0 to 10 P/B* | 5.0 P/B | 2.0 to 10 iB** | 5.0 iB |
| Formula 27 | 2.0 to 10 | 5.0 | 2.0 to 10 P/B* | 5.0 P/B | 2.0 to 10 iB** | 5.0 iB |
| Formula 28 | 2.0 to 10 | 5.0 | 2.0 to 10 P/B* | 5.0 P/B | 2.0 to 10 iB** | 5.0 iB |
| Formula 29 | 2.0 to 10 | 5.0 | 2.0 to 10 P/B* | 5.0 P/B | 2.0 to 10 iB** | 5.0 iB |
| Formula 30 | 2.0 to 10 | 5.0 | 2.0 to 10 P/B* | 5.0 P/B | 2.0 to 10 iB** | 5.0 iB |
| Formula 31 | 2.0 to 10 | 5.0 | 2.0 to 10 P/B* | 5.0 P/B | 2.0 to 10 iB** | 5.0 iB |
| Formula 32 | 2.0 to 10 | 5.0 | 2.0 to 10 P/B* | 5.0 P/B | 2.0 to 10 iB** | 5.0 iB |
| Formula 33 | 2.0 to 10 | 5.0 | 2.0 to 10 P/B* | 5.0 P/B | 2.0 to 10 iB** | 5.0 iB |
| Formula 34 | 2.0 to 10 | 5.0 | 2.0 to 10 P/B* | 5.0 P/B | 2.0 to 10 iB** | 5.0 iB |
| Formula 35 | 2.0 to 10 | 5.0 | 2.0 to 10 P/B* | 5.0 P/B | 2.0 to 10 iB** | 5.0 iB |
| Formula 36 | 2.0 to 10 | 5.0 | 2.0 to 10 P/B* | 5.0 P/B | 2.0 to 10 iB** | 5.0 iB |
| Formula 37 | 2.0 to 10 | 5.0 | 2.0 to 10 P/B* | 5.0 P/B | 2.0 to 10 iB** | 5.0 iB |
| Formula 38 | 2.0 to 10 | 5.0 | 2.0 to 10 P/B* | 5.0 P/B | 2.0 to 10 iB** | 5.0 iB |
| Formula 39 | 2.0 to 10 | 5.0 | 2.0 to 10 P/B* | 5.0 P/B | 2.0 to 10 iB** | 5.0 iB |
| Formula 40 | 2.0 to 10 | 5.0 | 2.0 to 10 P/B* | 5.0 P/B | 2.0 to 10 iB** | 5.0 iB |
| Formula 41 | 2.0 to 10 | 5.0 | 2.0 to 10 P/B* | 5.0 P/B | 2.0 to 10 iB** | 5.0 iB |
| Formula 42 | 2.0 to 10 | 5.0 | 2.0 to 10 P/B* | 5.0 P/B | 2.0 to 10 iB** | 5.0 iB |
| Formula 43 | 2.0 to 10 | 5.0 | 2.0 to 10 P/B* | 5.0 P/B | 2.0 to 10 iB** | 5.0 iB |
| Formula 44 | 2.0 to 10 | 5.0 | 2.0 to 10 P/B* | 5.0 P/B | 2.0 to 10 iB** | 5.0 iB |
| Formula 45 | 2.0 to 10 | 5.0 | 2.0 to 10 P/B* | 5.0 P/B | 2.0 to 10 iB** | 5.0 iB |
| Formula 46 | 2.0 to 10 | 5.0 | 2.0 to 10 P/B* | 5.0 P/B | 2.0 to 10 iB** | 5.0 iB |
| Formula 47 | 2.0 to 10 | 5.0 | 2.0 to 10 P/B* | 5.0 P/B | 2.0 to 10 iB** | 5.0 iB |
| Formula 48 | 2.0 to 10 | 5.0 | 2.0 to 10 P/B* | 5.0 P/B | 2.0 to 10 iB** | 5.0 iB |
| Formula 49 | 2.0 to 10 | 5.0 | 2.0 to 10 P/B* | 5.0 P/B | 2.0 to 10 iB** | 5.0 iB |
| Formula 50 | 2.0 to 10 | 5.0 | 2.0 to 10 P/B* | 5.0 P/B | 2.0 to 10 iB** | 5.0 iB |
| Formula 51 | 2.0 to 10 | 5.0 | 2.0 to 10 P/B* | 5.0 P/B | 2.0 to 10 iB** | 5.0 iB |
| Formula 52 | 2.0 to 10 | 5.0 | 2.0 to 10 P/B* | 5.0 P/B | 2.0 to 10 iB** | 5.0 iB |
| Formula 53 | 2.0 to 10 | 5.0 | 2.0 to 10 P/B* | 5.0 P/B | 2.0 to 10 iB** | 5.0 iB |
| Formula 54 | 2.0 to 10 | 5.0 | 2.0 to 10 P/B* | 5.0 P/B | 2.0 to 10 iB** | 5.0 iB |
| Formula 55 | 2.0 to 10 | 5.0 | 2.0 to 10 P/B* | 5.0 P/B | 2.0 to 10 iB** | 5.0 iB |
| Formula 56 | 2.0 to 10 | 5.0 | 2.0 to 10 P/B* | 5.0 P/B | 2.0 to 10 iB** | 5.0 iB |
| Formula 57 | 2.0 to 10 | 5.0 | 2.0 to 10 P/B* | 5.0 P/B | 2.0 to 10 iB** | 5.0 iB |
| Formula 58 | 2.0 to 10 | 5.0 | 2.0 to 10 P/B* | 5.0 P/B | 2.0 to 10 iB** | 5.0 iB |
| Formula 59 | 2.0 to 10 | 5.0 | 2.0 to 10 P/B* | 5.0 P/B | 2.0 to 10 iB** | 5.0 iB |
| Formula 60 | 2.0 to 10 | 5.0 | 2.0 to 10 P/B* | 5.0 P/B | 2.0 to 10 iB** | 5.0 iB |
| Formula 61 | 2.0 to 10 | 5.0 | 2.0 to 10 P/B* | 5.0 P/B | 2.0 to 10 iB** | 5.0 iB |
| Formula 62 | 2.0 to 10 | 5.0 | 2.0 to 10 P/B* | 5.0 P/B | 2.0 to 10 iB** | 5.0 iB |
| Formula 63 | 2.0 to 10 | 5.0 | 2.0 to 10 P/B* | 5.0 P/B | 2.0 to 10 iB** | 5.0 iB |
| Formula 64 | 2.0 to 10 | 5.0 | 2.0 to 10 P/B* | 5.0 P/B | 2.0 to 10 iB** | 5.0 iB |
| Formula 65 | 2.0 to 10 | 5.0 | 2.0 to 10 P/B* | 5.0 P/B | 2.0 to 10 iB** | 5.0 iB |
| Formula 66 | 2.0 to 10 | 5.0 | 2.0 to 10 P/B* | 5.0 P/B | 2.0 to 10 iB** | 5.0 iB |
| Formula 67 | 2.0 to 10 | 5.0 | 2.0 to 10 P/B* | 5.0 P/B | 2.0 to 10 iB** | 5.0 iB |
| Formula 68 | 2.0 to 10 | 5.0 | 2.0 to 10 P/B* | 5.0 P/B | 2.0 to 10 iB** | 5.0 iB |
| Formula 69 | 2.0 to 10 | 5.0 | 2.0 to 10 P/B* | 5.0 P/B | 2.0 to 10 iB** | 5.0 iB |
| Formula 70 | 2.0 to 10 | 5.0 | 2.0 to 10 P/B* | 5.0 P/B | 2.0 to 10 iB** | 5.0 iB |
| Formula 71 | 2.0 to 10 | 5.0 | 2.0 to 10 P/B* | 5.0 P/B | 2.0 to 10 iB** | 5.0 iB |
| Formula 72 | 2.0 to 10 | 5.0 | 2.0 to 10 P/B* | 5.0 P/B | 2.0 to 10 iB** | 5.0 iB |
| Formula 73 | 2.0 to 10 | 5.0 | 2.0 to 10 P/B* | 5.0 P/B | 2.0 to 10 iB** | 5.0 iB |
| Formula 74 | 2.0 to 10 | 5.0 | 2.0 to 10 P/B* | 5.0 P/B | 2.0 to 10 iB** | 5.0 iB |
| Formula 75 | 2.0 to 10 | 5.0 | 2.0 to 10 P/B* | 5.0 P/B | 2.0 to 10 iB** | 5.0 iB |

*“P/B” corresponds to a propane/butane mixture
**“iB” corresponds to isobutane

As explained above, the agents contemplated herein have advantageous hair-fixing properties. An exemplary embodiment therefore also provides a method for temporarily deforming keratinic fibers, in which a composition contemplated herein is applied onto the keratinic fibers. An exemplary embodiment also provides use of a cosmetic agent for temporarily deforming keratinic fibers. As explained above, the agents are distinguished in particular by improved hold in the case of temporary deformation of keratinic fibers. Another exemplary embodiment therefore additionally provides the use of a cosmetic agent to improve hold in the case of temporary deformation of keratinic fibers.

It has additionally been found that, by adding copolymer B to cosmetic agents containing copolymer A, the adhesive properties thereof are improved, in particular the tackiness and tacky phase (drying phase) thereof is reduced.

A further exemplary embodiment therefore further provides the use of a copolymer B formed from
a monomer (B1) selected from quaternized N-vinylimidazoles,
a monomer (B2) selected from N-vinyllactams,
   to reduce the tackiness of a cosmetic agent, containing in a cosmetically acceptable carrier a copolymer A formed from
a monomer (A1) from the group of unsaturated carboxylic acids and unsaturated carboxylic acid esters,
a monomer (A2) from the group of unsaturated amine group-containing monomers,
a monomer (A3) from the group of unsaturated amphiphilic monomers.

Yet another exemplary embodiment therefore provides the use of a copolymer B formed from
a monomer (B1) selected from quaternized N-vinylimidazoles,
a monomer (B2) selected from N-vinyllactams,
   to reduce the tacky phase of a cosmetic agent, containing in a cosmetically acceptable carrier a copolymer A formed from
a monomer (A1) from the group of unsaturated carboxylic acids and unsaturated carboxylic acid esters,
a monomer (A2) from the group of unsaturated amine group-containing monomers,
a monomer (A3) from the group of unsaturated amphiphilic monomers.

1. A cosmetic agent comprising:
a) a copolymer A formed from
a monomer (A1) from the group of unsaturated carboxylic acids and unsaturated carboxylic acid esters,
a monomer (A2) from the group of unsaturated amine group-containing monomers, and
a monomer (A3) from the group of unsaturated amphiphilic monomers
b) a copolymer B formed from
a monomer (B1) selected from quaternized N-vinylimidazoles, and
a monomer (B2) selected from N-vinyllactams, and
a cosmetically acceptable carrier.

2. The cosmetic agent according to claim 1, characterized in that copolymer A is formed from
a monomer (A1) from the group acrylic acid, methacrylic acid, C₁-C₆ alkyl acrylic acid esters, C₁-C₆ alkyl methacrylic acid esters,
a monomer (A2) from the group acrylamide, methacrylamide, mono-(C₁-C₄)-alkylamino-(C₁-C₄)-alkyl acrylate, di-(C₁-C₄)-alkylamino-(C₁-C₄)-alkyl acrylate, mono-(C₁-C₄)-alkylamino-(C₁-C₄)-alkyl methacrylate, di-(C₁-C₄)-alkylamino-(C₁-C₄)-alkyl methacrylate, and
a monomer (A3) from the group of C₁₀₋₃₀ alkyl PEG-20 itaconates.

3. The cosmetic agent according to claim 1, characterized in that a proportion by weight of copolymer A in the total weight of the cosmetic agent amounts to about 0.05 to about 10 wt. %.

4. The cosmetic agent according to claim 1, characterized in that copolymer B is formed from
3-methyl-1-vinylimidazolium methylsulfate (B1),
N-vinylpyrrolidone (B2a), and
N-vinylcaprolactam (B2b).

5. The cosmetic agent according to claim 1, characterized in that a proportion by weight of copolymer B in the total weight of the cosmetic agent amounts to about 0.05 to about 10 wt. %.

6. The cosmetic agent according to claim 1, characterized in that the weight ratio of copolymer A to copolymer B amounts to more than about 1:2.

7. The cosmetic agent according to claim 1, further comprising a monomeric quaternary ammonium compound.

8. The cosmetic agent according to claim 1, further comprising an oil body.

9. (canceled)

10. A method for temporarily deforming keratinic fibers, the method comprising the steps of:
applying a cosmetic agent to the keratinic fibers, the cosmetic agent comprising:
a copolymer A formed from
a monomer (A1) from the group of unsaturated carboxylic acids and unsaturated carboxylic acid esters,
a monomer (A2) from the group of unsaturated amine group-containing monomers, and
a monomer (A3) from the group of unsaturated amphiphilic monomers
a copolymer B formed from
a monomer (B1) selected from quaternized N-vinylimidazoles, and
a monomer (B2) selected from N-vinyllactams, and
a cosmetically acceptable carrier.

11. The cosmetic agent according to claim 3, characterized in that a proportion by weight of copolymer A in the total weight of the agent amounts to about 0.1 to about 7.0 wt. %.

12. The cosmetic agent according to claim 11, characterized in that a proportion by weight of copolymer A in the total weight of the agent amounts to about 0.2 to about 5.0 wt. %.

13. The cosmetic agent according to claim 5, characterized in that a proportion by weight of copolymer B in the total weight of the cosmetic agent amounts to about 0.1 to about 7.0 wt. %.

14. The cosmetic agent according to claim 13, characterized in that a proportion by weight of copolymer B in the total weight of the cosmetic agent amounts to about 0.2 to about 5.0 wt. %.

15. The cosmetic agent according to claim 6, characterized in that the weight ratio of copolymer A to copolymer B amounts to more than about 2:5.

16. The cosmetic agent according to claim 15, characterized in that the weight ratio of copolymer A to copolymer B amounts to more than about 1:3.

17. The cosmetic agent according to claim 16, characterized in that the weight ratio of copolymer A to copolymer B amounts to more than about 1:4.

18. The cosmetic agent according to claim 7, characterized in that the monomeric quaternary ammonium compound is selected from the group of trimethylalkylammonium halides.

19. The cosmetic agent according to claim 8, characterized in that the oil body is from the group of silicone oils.
